(19) United States
(12) Patent Application Publication
(10) Pub. No.: US 2013/0168267 A1
(43) Pub. Date: Jul. 4, 2013

(54) System and Method for Deodorizing Footwear

(71) Applicant: MVLE, Inc., Statesville, NC (US)

(72) Inventor: Pinch-Keeler April, Springfield, VA (US)

(73) Assignee: MVLE, INC., Statesville, NC (US)

(21) Appl. No.: 13/719,959

(22) Filed: Dec. 19, 2012

Related U.S. Application Data
(60) Provisional application No. 61/577,153, filed on Dec. 19, 2011.

(51) Int. Cl. A61L 9/00; B65B 1/04

(52) U.S. Cl. CPC A61L 9/00; B65B 1/04
USPC 206/5; 53/416

(57) ABSTRACT

The present invention is an odor nullifier for footwear comprising: a moisture absorbing fabric bag having a first and second fold, a vertical seam, an opening and a sealing member for sealing the opening, the fabric bag having a length and width for being received internally in a shoe; a scent carrier manufactured from a mixture consisting of 49% by weight of fine cut cellulose fiber, 49% by weight of granular vermiculite and 2% by weight of fragrant oil, the scent carrier disposed inside the fabric bag; wood shavings and dried flowers contained inside the fabric bag wherein the wood shavings are present in a quantity of at least 50% of the total weight.

### CLAIM OF PRIORITY

This patent application claims priory in U.S. Provisional Patent Application Ser. No. 61/577,153 filed Dec. 19, 2011.

### FIELD OF THE INVENTION

This invention is directed to the field of perfumery and odor control, and more specifically to a shoe freshener capable of being rejuvenated after use.

### BACKGROUND OF THE INVENTION

Odor generating from footwear is a common problem. One of the most common causes of foot odor is perspiration being deposited into the material of footwear. Perspiration contains water, salt, urea, and fatty acids. When these ingredients combine with microorganisms on the skin, the organisms can reproduce and grow in the footwear so that the footwear contains growing bacteria and fungus.

There have been many attempts to “deodorize” footwear such as U.S. Pat. No. 5,732,485 which discloses a deodorizer for masking foot and shoe odors having a retainer with an opening, a pressure-sensitive adhesive for securing the retainer to the interior surface of a shoe and a patch comprising a carrier, fragrance and polymer, wherein the fragrance is admixed with the polymer to retard evaporation or release of fragrance from the patch during storage and the carrier carries the fragrance and polymer. However, this invention requires that the deodorizer be installed in and remain in place even when the shoe is worn.

U.S. Pat. No. 4,228,549 attempts to solve the problem of footwear odor with socks or stockings of woven synthetic resin yarn in which activated charcoal is present in powder and small particles form in sufficient quantities to deodorize foot odors that may be present when the socks or stockings are worn.

United States Patent Application 2002/0083535 discloses a device for deodorizing and disinfecting a shoe by means of irradiation of biocidal ultraviolet (UV) light into the shoe interior. The device includes a substantially tubular light pipe that at one end incorporates a UV lamp connected to a power source, while the opposite end of the light pipe is inserted into a shoe. The inserted end may be covered by a protective cover fabricated of a thin polymer film that is substantially transparent in the UV spectral range. The light pipe has a reflective inner surface.

U.S. Pat. No. 7,047,671 discloses a disposable shoe insert that can include a deodorizer, such as a time-release fragrance, and/or odor neutralizer to help reduce or eliminate foot odor.

However, none of these inventions lend themselves to an easy-to-use device for managing foot odor in footwear or a device that can be rejuvenated once its effectiveness declines.

Therefore, it is an object of this invention to provide a system and method for controlling foot odor that is removable from the shoe when the shoe is worn and can be rejuvenated when effectiveness is lowered without having to add additional fragrance, deodorizer, and the like.

### SUMMARY OF THE INVENTION

The above objective is accomplished by providing an odor nullifier for footwear comprising: a moisture-absorbing fabric bag having a first and second fold, a vertical seam, an opening and a sealing member for sealing the opening, the fabric bag having a length and width for being received internally in a shoe; a scent carrier manufactured from a mixture consisting of 49% by weight of fine cut cellulose fiber, 49% by weight of granular vermiculite and 2% by weight of fragrant oil, the scent carrier disposed inside the fabric bag; wood shavings and dried flowers contained inside the fabric bag wherein the wood shaving are present in a quantity of at least 50% of the total weight.

The invention can also include dried flowers wherein the dried flowers are present in a quantity of at least 7.5% of the total weight. The dried flowers can weigh between 0.05 and 0.15 ounces. Further, the fabric bag can weigh between 0.10 and 0.30 ounces; the scent carrier can weigh between 0.30 and 0.40 ounces; and, the wood shavings weigh between 0.60 and 0.70 ounces.

The moisture regain of the fabric bag is between 7.1% and 8.5%. The fabric bag containing the scent carrier and the wood shavings weights between 1.0 and 1.6 ounces. The fabric bag can have a depth of about 1.5 inches, width of about 3.0 inches and length of about 5.0 inches so that the fabric bag, when received in the shoe, occupies at least 40% of the volume of the shoe cavity. The fabric bag fabric and the closing member can be 15.4% of the total weight; the scent carrier can be 26.9% of the total weight and the wood shavings are 50.0% of the total by weight.

The scent carrier can be taken from a mixture of 20 quarts of fine cut cellulose fiber, 20 quarts of granular vermiculate and 3½ cups of fragrant oil.

### DESCRIPTION OF THE DRAWINGS

The invention will be more readily understood from a reading of the following specifications and by reference to the accompanying drawings forming a part thereof, wherein an example of the invention is shown and wherein:

FIG. 1 is an elevation and perspective view of the invention;

FIG. 2 is elevation and perspective views of the invention;

FIG. 3 is a perspective view of the invention;

FIG. 4 is a elevation view of the invention;

FIG. 5 is a perspective view of the invention;

FIG. 6 is a perspective view of one side of the invention;

FIG. 7 is a perspective view of one side of the invention;

FIG. 8 is a side view of the operation on the invention; and,

FIG. 9 is a perspective view of the invention.

### DESCRIPTION OF A PREFERRED EMBODIMENT

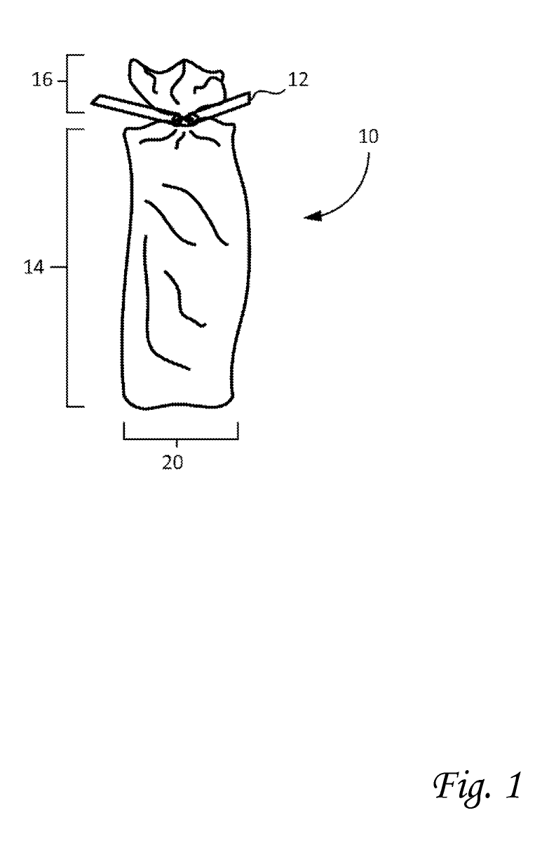

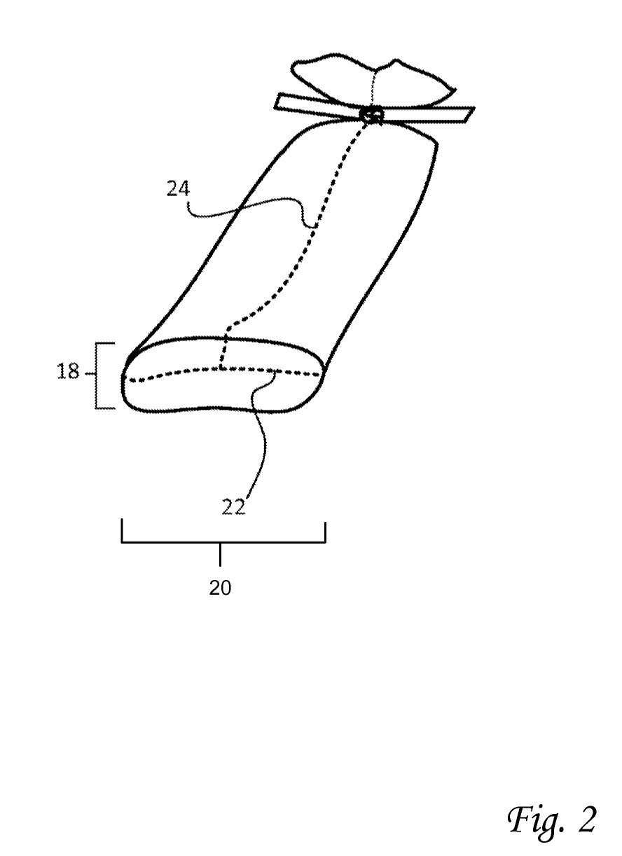

Referring to FIG. 1, a bag 10 is shown that has a lower section 14 that is about 6 inches in height to closing member 12 with an upper section 16 that is about 2 inches of material above the closing member. Referring to FIG. 2, the bag has a depth of about 1½ inches shown as 18 and a width of about 3 inches shown as 20. A bottom seam 22 and side seam 24 can exist in the bag where the sheet of fabric is sewn together to make the bag.

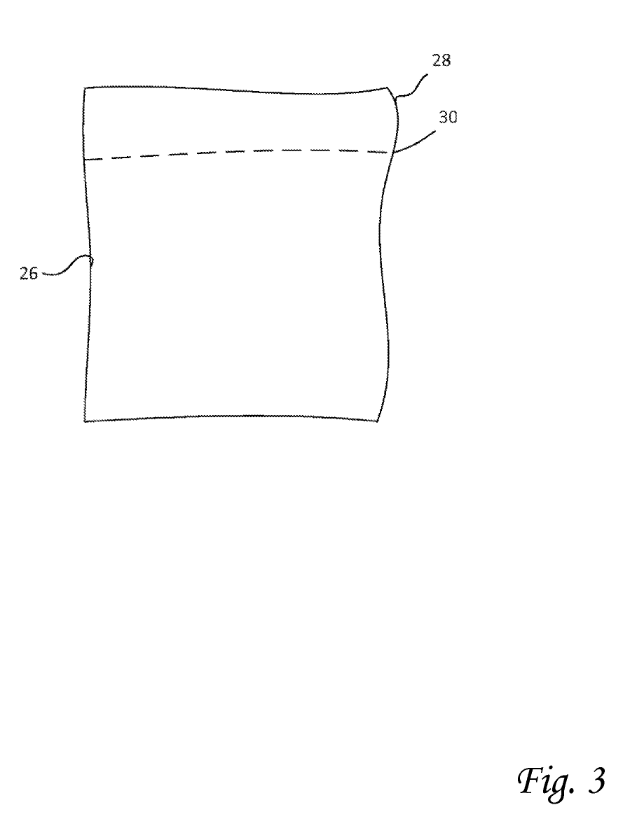

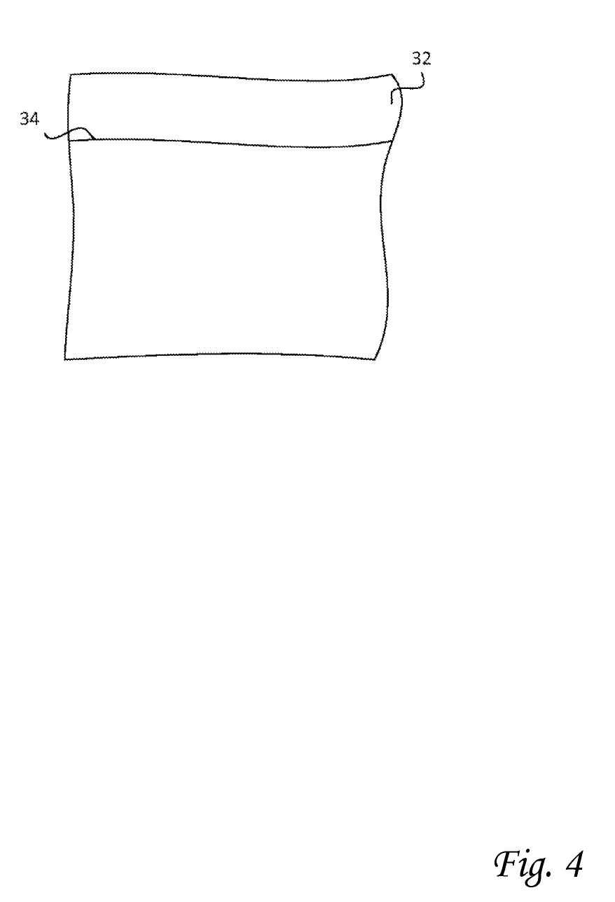

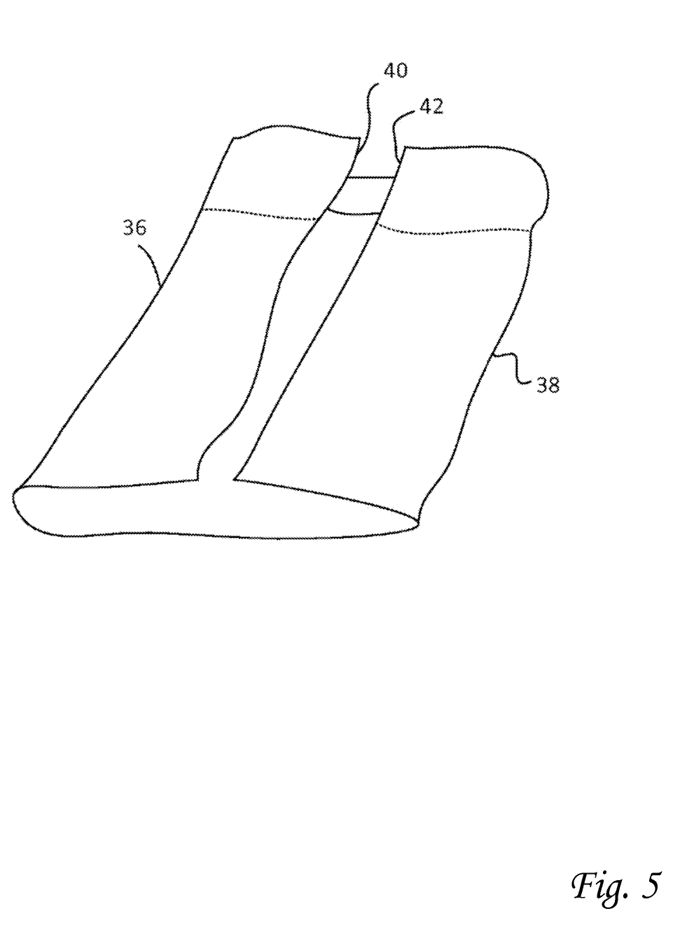

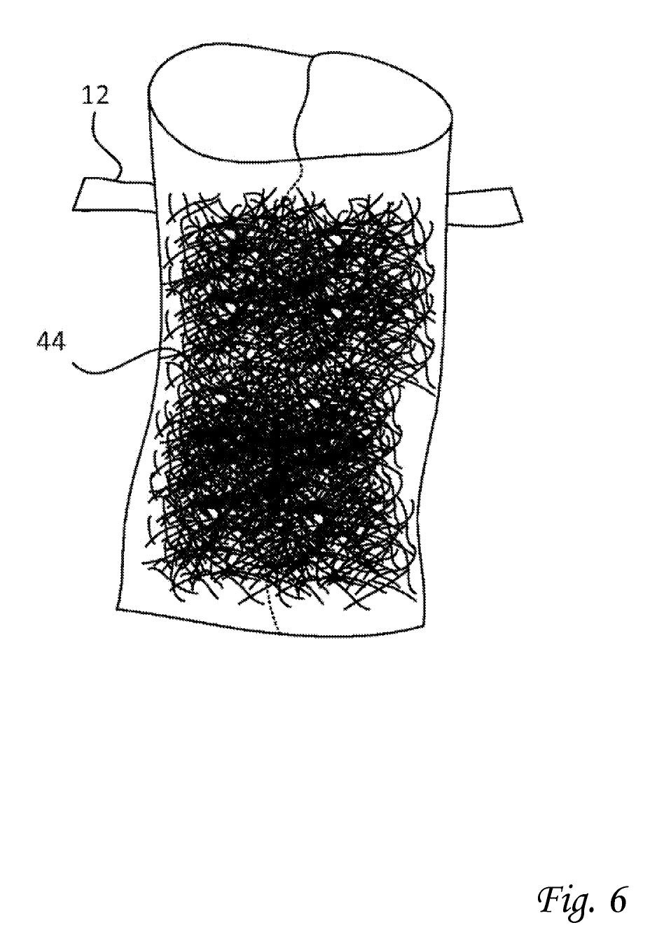

Referring to FIG. 3, a cut fabric 26 is used to make the bag. This fabric can be of any cloth material of synthetic material which allows air to penetrate the fabric. In one embodiment, the fabric is 10 by 10 inches. The top two inches 28 can be folded down along fold 30 so that the top 2 inches are reinforced (e.g., 2-ply). The top of the fabric can be sewn at seam 34 as shown in FIG. 4 to secure the top 2 inches in a folded arrangement making the fabric 10 by 8 inches. The fabric can then be folded lengthwise onto itself along folds 36 and 38 of FIG. 5 so that edges 40 and 42 can be sewn together. The bag can then be filled with the odor nullifying contents 44 as shown in FIG. 6 and closed with closing member 12.

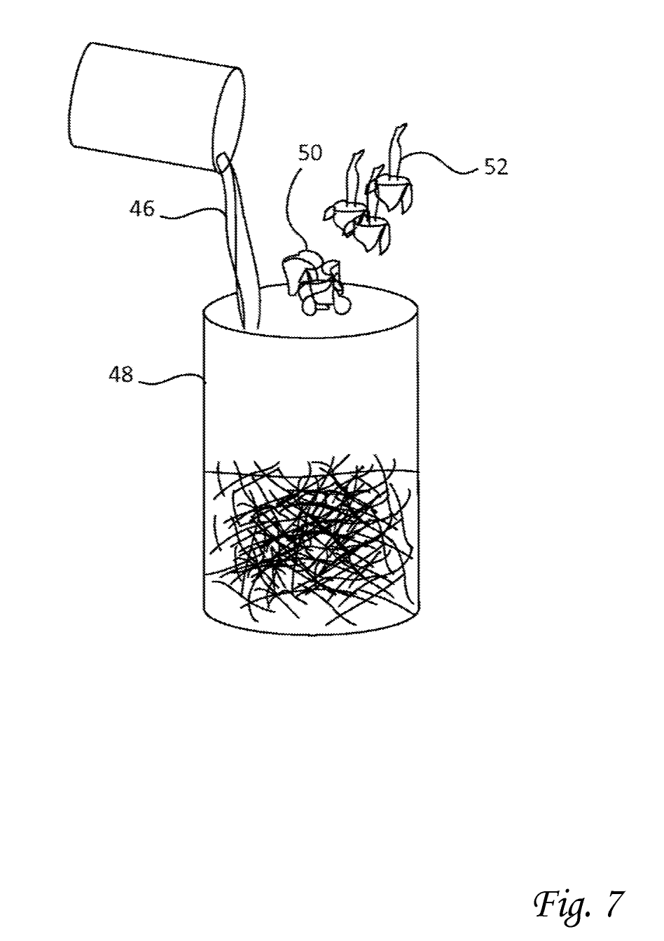

Referring to FIG. 7, the odor nullifying contents are described in more detail. In one embodiment, a scent carrier 46 is added to a mixing container 48. The scent carrier can be approximately 27% by weight of the invention. In one embodiment, 0.35 ounces of the scent carrier are used per bag. Cedar wood shavings 50 are added at approximately 50% by weight to the mixing container. Dried flowers 52 are added at approximately 7.5% by weight to the container.

In one embodiment, the scent carrier is a blend of approximately 50% fine cut cellulose fiber and 50% granular vermiculite. It has been discovered that use of vermiculite in this blend is highly absorbent and blends well with cellulose fiber with acceptable levels of resulting dust for the working and manufacturing environment. Blends or vermiculite in higher quantities leads to undesirable levels of dust production for the working and manufacturing environment.

In one embodiment, the scent carrier is 100% cut cellulose. In one embodiment, the scent carrier is approximately 49% fine cut cellulose fiber, 49% granular vermiculite, and 2% nautica premium fragrant oil. In one embodiment, the scent carrier is 48.93% fine cut cellulose fiber, 48.93% granular vermiculite, and 2.14% nautica premium fragrant oil. The nautica oil can be substituted with any desirable fragrant oil to obtain different scents. This composition results in a significantly stronger fragrance than that of the common household air freshener or even potpourri resulting in an effective odor nullifying agent. Further, since the cellulose absorbs the carrier scent, when the nullifying properties are less effective over time, the bags can be agitated (e.g., shaken, rolled between your hands, kneaded, otherwise compressed or subjected to friction), which results in more of the fragrance absorbed in the scent carrier being released, thereby rejuvenating the odor nullifying properties of the invention. The resulting composition has resulted in the useful life of the invention to exceed 6 months.

The odor nullifying content can be mixing in volume and subsequently divided and placed in individual bags. In one embodiment, the volume composition of the nullifying content includes 20 quarts of fine cut cellulose fiber, 20 quarts of granular vermiculite, and 3½ cups of nautica premium fragrant oil or a substituted fragrant oil.

In one embodiment, the invention comprises the following components by weight: between 12.3% and 18.5% bag fabric and closing member; 21.5% and 32.3% scent carrier; 40.0% and 60.0% wood shavings and 6.2% and 9.2% dried flower. The bag fabric can be a moisture-absorbing fabric for absorbing moisture from the interior of a shoe when the bag in placed in the shoe. In one embodiment, the moisture regain is between 7.1% and 8.5%. In one embodiment, the invention comprises the following components by weight: 15.4% bag fabric and closing member; 26.9% scent carrier; 50.0% wood shavings; and 7.7% dried flowers.

In one embodiment, the invention consists of 0.20 ounces of the fabric bag, 0.35 ounces of scent carrier, 0.65 ounces of cedar wood shavings, 0.10 ounces of dried flowers for a total weight of 1.30 ounces per bag. The bag can be of any fabric and can include designs such as a sport theme, flowers or any combination of designs as is common on fabrics. In one embodiment, the dried flowers can be eliminated and replaced with cedar wood shavings for 0.75 ounces. The closure member can be a ribbon of approximately 8 inches in length and ⅜ inches in width and can be double-sided satin.

It is important to note that the content mixture can vary by 33% or more so that precise measurements are not necessary for the invention to provide its utility. For example, scent carrier can be measured at 2 tablespoons, and the cedar wood shavings can be measured by two level cups. This would result in variations in the actual weight of the ingredients.

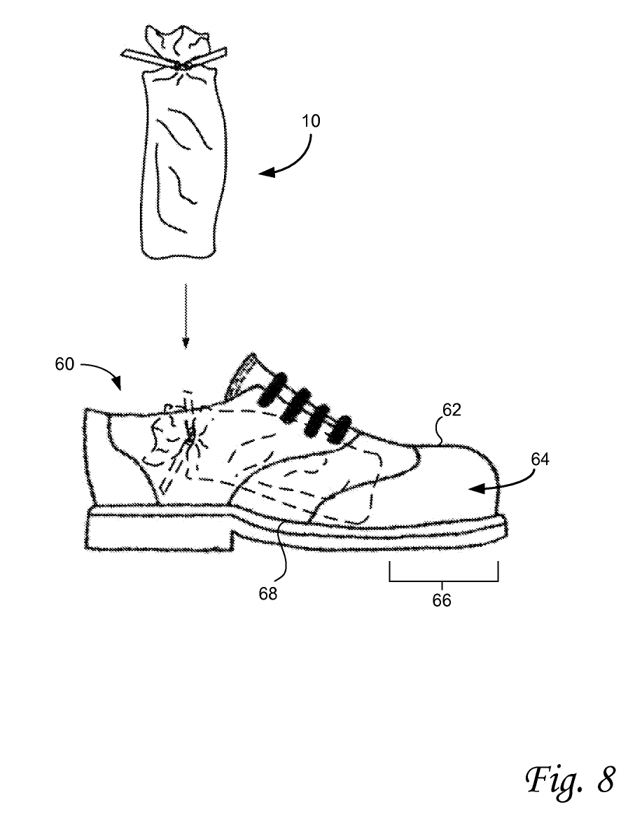

FIG. 8 shows the invention placed inside the shoe and functions to nullify odor. Bag 10 is sized to be received in shoe opening 60 of show 62. In one embodiment, the bag is partially inserted into the show defining a space 64 at the interior toe section 66 of the shoe. In one embodiment, the bag is in contact with the interior bottom 68 of the shoe. In this arrangement, the bag material can absorb moisture that is remaining in the shoe and facilitate with drying the shoe between uses. This functionality in combination with the odor neutralizing properties of the invention provide for a superior odor-fighting apparatus.

Typically, a shoe has a larger cross-section at the shoe opening than at the toe section. This is generally due to the shoe being designed to contour the shape and physiology of the human foot. The use of a fabric bag allows the bag to have a larger diameter at the shoe opening when inserted into the shoe and to compress due to the reduced cross-section of the shoe as the bag is received into the shoe.

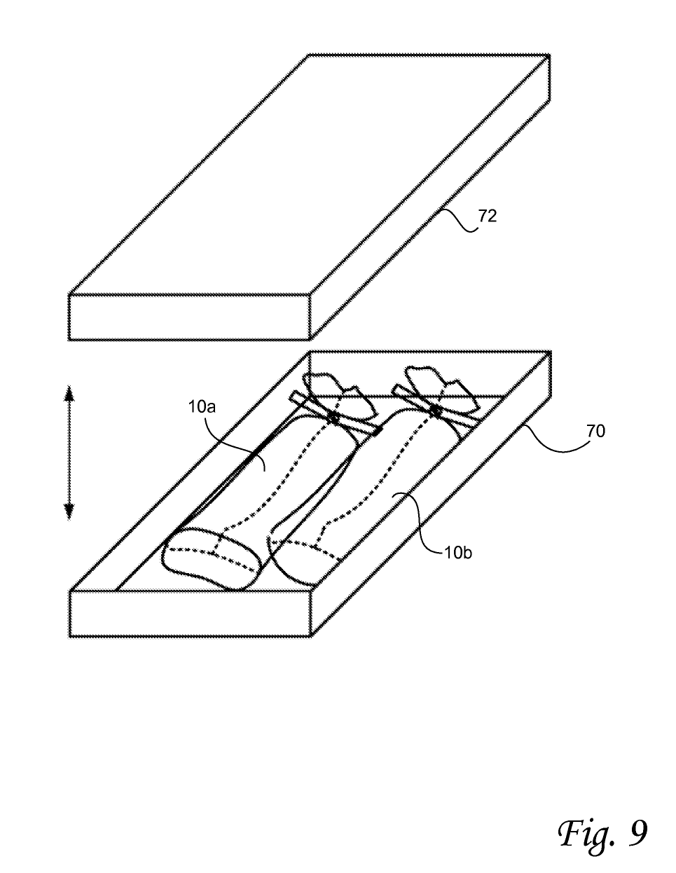

Referring to FIG. 9, the packaging for the invention is shown with two bags 10a and 10b arranged in a bottom container section 70 that can be enclosed with a top container section 72. The bags can be placed in a container such as a box approximately 5½ inches wide, 8 inches high and 2 inches deep. The box can be made from a top that has the top and side and a bottom that has a bottom and sides to better contain the fragrance when the bags are stored.

While the preferred embodiment of the invention has been illustrated and described, it will be appreciated that various changes can be made therein without departing from the spirit and scope of the invention.

What is claimed is:

1. An odor nullifier for footwear comprising:
a moisture absorbing fabric bag having a first and second fold, a vertical seam, an opening and a sealing member for sealing said opening, said fabric bag having a length and width for being received internally in a shoe;
a scent carrier manufactured from a mixture consisting of 49% by weight of fine cut cellulose fiber, 49% by weight of granular vermiculite and 2% by weight of fragrant oil, said scent carrier disposed inside said fabric bag;
wood shavings and dried flowers contained inside said fabric bag wherein said wood shavings are present in a quantity of at least 50% of the total weight.

2. The apparatus of claim 1 including dried flowers wherein said dried flowers are present in a quantity of at least 7.5% of the total weight.

3. The apparatus of claim 2 wherein said dried flowers weigh between 0.05 and 0.15 ounces.

4. The apparatus of claim 1 wherein:
said fabric bag weights between 0.10 and 0.30 ounces;
said scent carrier weights between 0.30 and 0.40 ounces; and,
said wood shavings weigh between 0.60 and 0.70 ounces.

5. The apparatus of claim 1 wherein the moisture regain of said fabric bag is between 7.1% and 8.5%.

6. The apparatus of claim 1 wherein said fabric bag containing said scent carrier and said wood shavings weighs between 1.0 and 1.6 ounces.

7. The apparatus of claim 1 wherein said fabric bag has a depth of about 1.5 inches, width of about 3.0 inches and length of about 5.0 inches so that said fabric bag, when received in said shoe, occupies at least 40% of the volume of the cavity defined in said shoe.

8. The apparatus of claim 1 wherein said fabric bag fabric and said closing member is 15.4% of the total weight; said scent carrier is 26.9% of the total weight and said wood shavings are 50.0% of said total by weight.

9. The apparatus of claim 1 wherein said scent carrier is taken from a mixture of 20 quarts of fine cut cellulose fiber, 20 quarts of granular vermiculate and 3½ cups of fragrant oil.

10. An odor nullifier for footwear comprising:
a moisture absorbing fabric bag having a first and second fold, a vertical seam, an opening and a sealing member for sealing said opening, said fabric bag having a length and width for being received internally in a shoe to occupy at least 40% of the cavity defined in said shoe;
a scent carrier manufactured from a mixture consisting of less than 98% by weight fine cut cellulose fiber and at least 2% by weight of fragrant oil carried inside said fabric bag; and,
wood shavings and dried flowers contained inside said fabric bag wherein said wood shaving are present in a quantity of at least 50% of the total weight and said dried flowers present in a quantity of at least 7.5% of the total weight.

11. The apparatus of claim 10 wherein said scent carrier includes between 0% and 50% by weight of granular vermiculite.

12. The apparatus of claim 10 wherein said scent carrier is taken from a mixture of 20 quarts of fine cut cellulose fiber and 3½ cups of fragrant oil.

13. The apparatus of claim 12 wherein said mixture includes 20 quarts of granular vermiculite.

14. The apparatus of claim 10 wherein said scent carrier includes 48.93% by weight of fine cut cellulose fiber.

15. The apparatus of claim 10 wherein said scent carrier includes 48.93% by weight of granular vermiculite.

16. The apparatus of claim 10 wherein said scent carrier includes 2.14% by weight of fragrant oil.

17. A method of nullifying odor in a shoe comprising the steps of:
providing a moisture absorbing fabric bag having a moisture regain of between 7.1% and 8.5%;
placing a scent carrier consisting of 98% by weight of fine cut cellulose fiber and at least 2% by weight of fragrant oil;
placing wood shavings and dried flowers inside said fabric bag wherein said wood shavings are present in a quantity of at least 50% of the total weight; and,
sealing said fabric bag with a sealing member.

18. The method of claim 17 including placing granular vermiculite inside said fabric bag in a quantity of 50% or less of the total weight.

19. The method of claim 17 including providing two fabric bags containing said scent carrier, wood shavings and said dried flowers for use with a pair of shoes.

20. The method of claim 17 including a two-ply layer included in said fabric bag disposed at an opening at said fabric bag.
